(19) United States
(12) Patent Application Publication
(10) Pub. No.: US 2023/0210785 A1
(43) Pub. Date: Jul. 6, 2023

(54) COMPOSITIONS COMPRISING CAROTENOIDS, METHODS AND APPLICATION THEREOF

(71) Applicant: Biogen Extracts Pvt Ltd, Bangalore (IN)

(72) Inventor: Mohammed Ali Salman Mehkri, Bangalore (IN)

(73) Assignee: Biogen Extracts Pvt Ltd, Bangalore (IN)

(21) Appl. No.: 18/146,393

(22) Filed: Dec. 26, 2022

(30) Foreign Application Priority Data
Dec. 25, 2021 (IN) 202141060758

(51) Int. Cl. A61K 31/047; A61K 31/01; A61K 31/015; A61K 47/44; A61K 31/355; A61K 36/537; A61K 47/36; A61P 27/02

(52) U.S. Cl. CPC A61K 31/047; A61K 31/01; A61K 31/015; A61K 31/355; A61K 36/537; A61K 47/36; A61K 47/44; A61P 27/02; A61K 2236/37

(57) ABSTRACT

Invention deals with a macular carotenoid formulation comprising Carotenoids, Lutein, Zeaxanthin, Meso Zeaxanthin, Beta Carotene, Alpha Carotene, Lycopene and Cryptoxanthin in a synergistic combination. The formulation helps in increasing macular pigment optical density (MPOD) related to prevention of age-related macular degeneration (ARMD). The formulation is used as a nutrient, nutraceutical or dietary supplement. The dietary supplement may be formulated as soft gel capsules, two-piece hard-shell capsules, liquid-fill capsules, tablets, effervescent granules, gummies, powder mixes, stick packs, beverages, emulsions, bakery products, dairy products, tinctures, oil suspensions, bead-lets, powders, cold water-soluble powders, emulsions and granules or any combination thereof. The present invention can be used for the development of functional food, dietary plan and as a nutritional supplement. In exemplary embodiment, formulation is available in forms like oil suspension, beadlets, powders, cold water-soluble powders, emulsions and granules.

### CROSS-REFERENCE TO RELATED APPLICATIONS

This application claims priority from Indian provisional application 202141060758 filed on Dec. 25, 2021 titled “COMPOSITIONS COMPRISING CAROTENOIDS, METHODS AND APPLICATION THEREOF”

### TECHNICAL FIELD OF THE INVENTION

The invention relates generally to carotenoid formulations and, more particularly, to a synergistic macular carotenoid formulation for increasing the macular pigment optical density (MPOD) levels and central foveal thickness (CFT) to support healthy vision in subjects.

### BACKGROUND OF THE INVENTION

Globally, it is seen that age-related ophthalmic diseases such as cataracts, age-related macular degeneration (AMD), glaucoma, and diabetic retinopathy are the main causes of progressive and irreversible vision loss. However, pathogenic processes related to these diseases are complex and unclear, and may depend on numerous factors which include continuous exposure to blue light, which is quite evident with the usage of mobile devices and screen time associated with it. Unfortunately, most of these conditions are diagnosed in advanced stages at which effective treatments are not available.

The macula located in the inner retina of the eye is denatured over a period due to aging, genetic factors, toxicity, inflammation, etc., resulting in decreased visual acuity and severe loss of vision. In the past, most patients with macular degeneration were mostly age-related macular degeneration (AMD). The current trend shows that young people are diagnosed with macular degeneration, and it is on the rise. Macular degeneration in younger people is currently attributed to the damage caused by blue light. Blue light has a short wavelength incorporated in the LED of various electronic screens such as computers and smartphones have high energy. When one is exposed to blue light continuously, reactive oxygen species (ROS) are increased in retinal pigment epithelium (RPE) cells, leading to macular degeneration. These degenerated macular cells lead to clinical symptoms which can be recognized that effect vision and acuity. Unfortunately, these conditions are irreversible and may have a long-lasting impact on eye health.

Macular pigment levels peak at the central foveal area (predominantly found in the retina), which is measured through the central foveal thickness. Macular pigment is composed of two dietary carotenoids, lutein and zeaxanthin, and is mainly present at the nerve fiber layers and ganglion cell layers of the retina, with peak concentrations in the fovea. It is thought to function as a blue-light filter and antioxidant, and therefore protect the retina from damaging influences that are thought to play a role in the pathogenesis of age-related macular degeneration.

The key symptom associated with macular degeneration is the loss in the center of the field of vision leading to blurred vision. In dry macular degeneration, the center of the retina deteriorates. In addition, in wet macular degeneration, leaky blood vessels grow under the retina. a diet supplemented with antioxidants can be a part of a defense strategy to minimize oxidative damage in a vulnerable population such as the elderly. Age-Related Eye Disease Studies (AREDS) found that supplementation with certain vitamins and minerals can slow the progression of intermediate to advanced age-related macular degeneration (AMD).

Formulas have been developed as dietary supplements to cater to the high demand from consumers to support healthy vision and prevent eye problems. Vitamins and minerals in dietary supplements have been scientifically established for reducing the risks of several diseases, including macular degeneration and other eye-related diseases. However, knowledge of the right formulation with the right dosage, efficacy, and bioavailability of antioxidants, vitamins, and minerals is not available to consumers.

So, there is a need in the art to develop a formulation with dietary supplements and nutraceuticals following the evidence-based recommended dosages and reference intakes for improving general health and preventing eye-related diseases.

### SUMMARY OF THE INVENTION

The primary objective of the present invention is to provide a macular carotenoid formulation comprising Lutein, Zeaxanthin isomers, Beta Carotene, Alpha Carotene, Lycopene and Cryptoxanthin in a synergistic combination. The formulation helps in increasing macular pigment optical density (MPOD) and central foveal thickness (CFT). The formulation of the present invention helps to prevent age-related macular degeneration (ARMD) and protect against oxidative damage and blue light, thereby supporting healthy vision.

According to an aspect of the invention, a macular carotenoid formulation for increasing the macular pigment optical density (MPOD) level in a subject is provided. The composition comprises a) Lutein in an amount of 2 to 20 mg, b) Zeaxanthin isomers in an amount of 0.4 to 4 mg, c) Beta Carotene in an amount of 1 to 10 mg, d) Alpha Carotene in an amount of 40 to 1000 mcg, e) Lycopene in an amount of 1 to 10 mg, and f) Beta Cryptoxanthin in an amount of 1 to 1000 mcg.

In an embodiment, the macular carotenoid formulation is formulated using a carrier oil selected from sunflower oil, safflower oil, MCT (Medium Chain Triglycerides), castor oil, soybean oil, corn oil, olive oil Omega-3 fatty acid oil or combinations thereof.

In another embodiment, the formulation comprises at least one stabilizing agent selected from gum arabic, modified food starch, maltodextrin, cyclodextrin, sucrose, lecithin or combinations thereof.

In yet another embodiment, the formulation is used as a nutrient, nutraceutical or dietary supplement.

According to another aspect of the invention, a macular carotenoid formulation for increasing the macular pigment optical density (MPOD) level in a subject is provided. The composition comprises: a) from about 1.66% to 16.6% by weight of Lutein, b) from about 0.34% to 3.4% by weight of Zeaxanthin-isomers, c) from about 0.74% to 8% by weight of Beta Carotene, d) from about 0.01% to 0.4% by weight of Alpha Carotene, e) from about 0.6% to 6.2% by weight of Lycopene, f) from about 0.001% to 0.1% by weight of Beta Cryptoxanthin, g) from about 0.01% to 2% by weight of Antioxidant, and h) from about 50% to 90% by weight of Carrier Oil.

In an embodiment, the anti-oxidant is selected from vitamin E, mixed Vitamin E, mixed tocopherols or rosemary extract or combinations thereof and the carrier oil is selected from sunflower oil, safflower oil, MCT (Medium Chain Triglycerides), castor oil, soybean oil, corn oil, olive oil, Omega-3 fatty acid oil or combinations thereof.

According to yet another aspect of the invention, a macular carotenoid formulation for increasing the macular pigment optical density (MPOD) level in a subject is provided. The composition comprises a) from about 1.66% to 16.7% by weight of Lutein, b) from about 0.34% to 3.4% by weight of Zeaxanthin-isomers, c) from about 0.74% to 8% by weight of Beta Carotene, d) from about 0.01% to 0.4% by weight of Alpha Carotene, e) from about 0.6% to 6.2% by weight of Lycopene, f) from about 0.001% to 0.1% by weight of Beta Cryptoxanthin, g) from about 0.01% to 2% by weight of Antioxidant, h) from about 10% to 30% by weight of gum Arabic, and i) from about 25% to 75% by weight of a stabilizing agent.

In an embodiment, the anti-oxidant is selected from vitamin E, mixed Vitamin E, mixed tocopherols or rosemary extract Or combinations thereof.

In another embodiment, the stabilizing agent comprises at least one of a gum arabic, modified food starch, maltodextrin, cyclodextrin, sucrose, lecithin and combinations thereof.

In yet another embodiment, the composition comprises a) about 8.9% by weight of Lutein, b) about 1.8% by weight of Zeaxanthin-isomers, c) about 5.2% by weight of Beta Carotene, d) about 0.3% by weight of Alpha Carotene, e) about 3.1% by weight of Lycopene, f) about 0.1%, by weight of Beta Cryptoxanthin, g) about 1.0% by weight of Antioxidant, h) about 20.5% by weight of gum Arabic, and i) about 59.0% by weight of modified food starch.

In yet another embodiment, the composition comprising the Lutein, the Zeaxanthin-isomers, the Beta Carotene, the Alpha Carotene, the Lycopene, the Beta Cryptoxanthin are stabilized in the gum arabic and/or the modified food starch along with the antioxidant selected from the group comprising vitamin E, mixed Vitamin E, mixed tocopherols and rosemary extract.

In yet another embodiment, the formulation is formulated as soft gel capsules, two-piece hard-shell capsules, liquid-fill capsules, tablets, effervescent granules, gummies, powder mixes, stick packs, beverages, emulsions, bakery products, dairy products, tinctures, oil suspensions, bead-lets, powders, cold water-soluble powders, emulsions and granules or any combination thereof.

In yet another embodiment, the subject is a mammal.

In yet another embodiment, the subject is a human.

In yet another embodiment, a method of preparing the macular carotenoid formulation is provided. The macular carotenoid composition is derived from the vegetarian sources comprising xanthophylls. The Lutein, Zeaxanthin and Meso Zeaxanthin are extracted from marigold flowers (Tagetes erecta), Alpha Carotene from carrot (Daucus carota subsp. sativus), the Beta Carotene is extracted from Blakeslea trispora, and the Lycopene is extracted from Blakeslea trispora.

In yet another embodiment, the Beta Carotene is obtained as a synthetic extraction.

In yet another embodiment, the Beta Carotene is extracted from Dunaliella salina and the Alpha carotene is extracted from Elaeis guineensis.

In yet another embodiment, the Lycopene is extracted from Solanum lycopersicum.

In yet another embodiment, the Lutein, Zeaxanthin and Meso Zeaxanthin, Alpha carotene, Beta carotene and Lycopene are derived from the vegetarian sources using solvent extraction.

In yet another embodiment, the Lutein, Zeaxanthin and Meso Zeaxanthin, Alpha Carotene, Beta Carotene and Lycopene are derived from the vegetarian sources using super-critical extraction.

Several aspects of the invention are described below with reference to examples for illustration. However, one skilled in the relevant art will recognize that the invention can be practiced without one or more of the specific details or with other methods, components, materials and so forth. In other instances, well-known structures, materials, or operations are not shown in detail to avoid obscuring the features of the invention. Furthermore, the features/aspects described can be practiced in various combinations, though only some of the combinations are described herein for conciseness.

### BRIEF DESCRIPTION OF THE DRAWINGS

Example embodiments of the present invention will be described with reference to the accompanying drawings briefly described below.

FIG. 1A illustrates a flow chart of a method of preparing the macular carotenoid formulation in the form of oil suspension according to the aspect of the present invention.

FIG. 1B illustrates a flow chart of a method of preparing the macular carotenoid formulation in the form of granules according to the aspect of the present invention.

FIG. 2 illustrates improvement in MPOD levels in subjects treated with test and placebo between Visit 1 to Visit 4, according to the aspect of the present invention.

FIG. 3 illustrates a graphical representation of improvement in Central Foveal Thickness (CFT), according to the aspect of the present invention.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE INVENTION

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another.

As used herein, the singular forms “a”, “an”, and “the” include both singular and plural referents unless the context clearly dictates otherwise. By way of example, “a dosage” refers to one or more than one dosage.

The terms “comprising”, “comprises” and “comprised of” as used herein are synonymous with “including”, “includes” or “containing”, “contains”, and are inclusive or open-ended and do not exclude additional, non-recited members, elements or method steps.

All documents cited in the present specification are hereby incorporated by reference in their totality. In particular, the teachings of all documents herein specifically referred to are incorporated by reference.

Example embodiments of the present invention are described with reference to the accompanying figures.

In the drawings, like reference numbers generally indicate identical, functionally similar, and/or structurally similar elements. The drawing in which an element first appears is indicated by the leftmost digit(s) in the corresponding reference number.

### Definitions

“Macular Pigment Optical Density (MPOD)” refers to a measure of the density of Macular Pigment (MP) in the center of the retina.

“Age-Related Macular Degeneration (ARMD)” refers to an eye disease that occurs when aging causes damage to the macula which is a part of the eye that controls sharp, straight-ahead vision.

“Central Foveal Thickness (CFT)” refers to the mean thickness measured at the point of intersection of the 6 radial scans on optical coherence tomography.

“Macular Assessment Profile (MAP) test” refers to a test that measures the spatial distribution of Macular Pigment in the eye.

“VDU-based test” refers to the examination of the vision under optimally simulated VDU (Visual Display Unit) working conditions.

“Optical coherence tomography (OCT)” refers to a non-invasive imaging test. OCT uses light waves to take cross-section pictures of the retina.

“Central Fovea” refers to a small depression within the neurosensory retina where visual acuity is the highest.

“Snellen Visual Acuity” refers to an eye chart that can be used to measure visual acuity.

“Carotenoids” refers yellow, orange, and red organic pigments that are produced by plants and algae, as well as several bacteria, and fungi.

“Lutein”, “Alpha Carotene”, “Beta Carotene”, “Beta Cryptoxanthin”, “Lycopene”, “Zeaxanthin” refer to a naturally occurring carotenoid.

“Zeaxanthin isomers” refers to the isomeric distribution of zeaxanthin denoted to as zeaxanthin and meso-zeaxanthin.

“Carrier oil” refers to a base oil or vegetable oil used to dilute essential oils and absolutes.

“Stabilizing agent” refers to compounds that are added to food products to provide and preserve structure, stability, and viscosity.

“Antioxidants” refers to chemicals that lessen or prevent the effects of free radicals.

“Gum arabic” refers to a gum that contains polysaccharides.

“Modified food starch” refers to a physically, enzymatically, or chemically altered starch with changed inherent properties.

“Maltodextrin” refers to a polysaccharide produced from starchy substances.

“Cyclodextrin” refers to cyclic oligosaccharides consisting of a macrocyclic ring of glucose subunits joined by α-1,4 glycosidic bonds.

“Sucrose” refers to a molecule composed of two monosaccharides, namely glucose and fructose.

“Lecithin” refers to yellow-brownish fatty substances occurring in animal and plant tissues that are amphiphilic.

“Emulsion” refers to a mixture of two or more liquids that are normally immiscible owing to liquid-liquid phase separation.

“Tincture” refers to an extract of plant material dissolved in ethanol (ethyl alcohol).

“Effervescent granules” are granules, or coarse to very coarse powders, containing the medicinal agent in a dry mixture usually composed of sodium bicarbonate, citric acid, and tartaric acid.

“Synthetic extraction” refers to a process of extraction using synthetic extraction chemicals.

“Solvent extraction” refers to a process in which a compound transfers from one solvent to another owing to the difference in solubility or distribution coefficient between the two immiscible (or slightly soluble) solvents.

“Supercritical fluid extraction (SFE)” refers to a process of separating one component (the extractant) from another (the matrix) using supercritical fluids as the extracting solvent.

### Embodiments of the Invention

The primary objective of the present invention is to provide a novel formulation, comprising a mixed carotenoid complex for its activity in increasing macular pigment optical density (MPOD), related to the prevention of age-related macular degeneration (ARMD).

The central portion of the retina or macula is responsible for optimal spatial vision. Macular pigment (MP) is a term used to describe the yellow pigment composed principally of the three isomeric carotenoids lutein, meso-zeaxanthin and zeaxanthin, which accumulate in the macula. There is increasing evidence that MP is important for vision in normal subjects. since oxidative damage seems to be an important factor for the development and exacerbation of some retinal diseases, the postulated protective role of MP in some disorders, especially Age-Related Macular Degeneration (AMD), has been extensively investigated over the past decades.

These carotenoids work as a filter protecting the macula from blue light and as a resident antioxidant and free radical scavenger to reduce oxidative stress-induced damage. Many observational and interventional studies have suggested that lutein and zeaxanthin may reduce the risk of various eye diseases, especially late forms of AMD. In vitro and in vivo studies indicate that they could protect various ocular cells against oxidative damage.

Lutein, meso-zeaxanthin and zeaxanthin are possibly combined with other carotenoids in a synergistic combinaion. This invention provides a formulation of lutein, meso-zeaxanthin and zeaxanthin with other carotenoids showing higher efficacy in improving MPOD than lutein, meso-zeaxanthin and zeaxanthin alone.

### EXAMPLE EMBODIMENTS

The present invention is illustrated in further details by the following non-limiting examples.

### Example 1: Components of the Composition

The results of observational studies suggest that diets high in carotenoid-rich fruits and vegetables are associated with reduced risks of cardiovascular disease and some cancers. All carotenoids, such as lutein, zeaxanthin, beta carotene, Alpha carotene, Beta cryptoxanthin and lycopene have been studied individually for their health benefits. However, a synergistic approach on managing overall health has not been considered.

The said formulation aims to resolve this gap by conducting several clinical studies from a holistic approach to well-being. The first study conducted was on eye-health (MPOD), where the said formulation demonstrated higher levels than lutein & zeaxanthin isomers alone.

According to an embodiment, the macular carotenoid formulation comprises,
Lutein - 10 mg (5 mg to 20 mg)
Zeaxanthin-isomers - 2 mg (1 mg to 4 mg)
Beta Carotene - 5.7 mg (2.85 mg to 5.7 mg)
Alpha Carotene - 240 mcg (120 mcg to 240 mcg)
Lycopene - 5 mg (2.5 mg to 7.5 mg)
Beta Cryptoxanthin - Traces

### Example 2: Method of Preparing the Formulation

The carotenoids are derived from the vegetarian sources comprising xanthophylls. The Lutein, Zeaxanthin and Meso Zeaxanthin are extracted from marigold flowers (Tagetes erecta), Alpha Carotene is extracted from carrot (Daucus carota subsp. Sativus or Elaeis guineensis), the Beta Carotene is extracted from Blakeslea trispora or Dunaliella salina, the Lycopene is extracted from Blakeslea trispora or Solanum lycopersicum. Each component of the formulation is individually extracted from their respective raw materials using solvent extraction or super-critical extraction. The Solvent extraction is performed using Ethanol, methanol, iso propyl alcohol, isobutyl alcohol, ethyl acetate or combinations thereof. The solvent extraction is performed for about 2 to 8 hours at a temperature ranging from 40 to 85° C. The supercritical extraction is performed using carbon-di-oxide as a supercritical fluid. The supercritical extraction is performed at a temperature ranging from 30 to 60° C. with a duration of about 2 hours to 8 hours.

After extraction, the components of the formulation comprising Lutein, Zeaxanthin isomers, Beta Carotene, Alpha Carotene, Lycopene and Beta Cryptoxanthin are homogenized in a carrier oil at room temperature to form the oil suspension. The carrier oil is selected from sunflower oil, safflower oil, MCT (Medium Chain Triglycerides), castor oil, soybean oil, corn oil, olive oil, Omega-3 fatty acid oil or combinations thereof.

According to an embodiment, to obtain the formulation in the form of granules, the components of the formulation comprising Lutein, Zeaxanthin isomers, Beta Carotene, Alpha Carotene, Lycopene and Beta Cryptoxanthin are stabilized in gum Arabic, modified food starch maltodextrin, cyclodextrin, sucrose, lecithin and combinations thereof along with an antioxidant such as vitamin E or mixed tocopherols or rosemary extract.

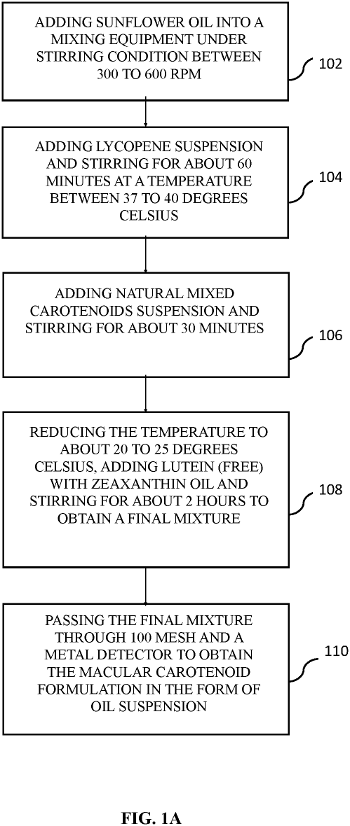

FIG. 1A illustrates a flow chart of a method of preparing the macular carotenoid formulation in the form of oil suspension according to the aspect of the present invention. At step 102, sunflower oil is added into a mixing equipment under stirring condition between 300 to 600 rpm. At step 104, Lycopene Suspension is added and stirred for about 60 minutes at a temperature between 37 to 40° C. At a step 106, Natural Mixed Carotenoids Suspension is added and stirred for about 30 minutes. At step 108, the temperature is reduced to about 20 to 25° C., and Lutein (Free) with Zeaxanthin Oil is added and stirred for about 2 hours to obtain a final mixture. At step 110, the final mixture is passed through 100 Mesh and Metal Detector to obtain the macular carotenoid formulation in the form of oil suspension.

FIG. 1B illustrates a flow chart of a method of preparing the macular carotenoid formulation in the form of granules. At step 112, the components of the formulation comprising Lutein, Zeaxanthin isomers, Carotene, Alpha Carotene, Lycopene are obtained powder form and Blended in a Blender. At step 114, a solvent comprising 10% - 30% by weight is sprayed in the Blender to obtain a mixture. At step 116, the mixture is added onto a 10 Mesh Granulator to obtain granules. At step 118, the granules are separated based on particle size using 30 Mesh and 80 Mesh. At step 120, the separated granules are blended in the blender. At step 122, the blended granules are Pass through a metal detector to obtain the macular carotenoid formulation in the form of granules.

### Example 3: Efficacy of the Formulation on MPOD

A randomized, double blind, placebo-controlled, parallel study to evaluate the comparative safety and comparative efficacy of the formulation, and to assess any positive correlation between MPOD and central foveal thickness levels, in healthy adult subjects.

Test Arm - 35 subjects receiving a half dose of Test product 1 (Lutein 5 mg + Zeaxanthin 1 mg) or Test product 2 comprising the macular carotenoid formulation of the present invention (Lutein 5 mg + Zeaxanthin 1 mg + Natural Mixed Carotenoids 5.5 mg) twice daily in softgel capsules over 180 days.

Placebo - 23 subjects receiving sunflower oil twice daily in softgel capsules over 180 days.

### Example 4: Clinical Study of the Efficacy of The Formulation

Purpose: To assess Macular Pigment Optical Density (MPOD) levels and compare them with the placebo as well as establish the positive correlation between Central Foveal Thickness (CFT) and MPOD levels in healthy adult subjects.

Method: It was a randomized, double blind, placebo controlled, parallel, three arm study in healthy adult subjects. The participants underwent general physical and ophthalmic examinations. MPOD was measured using the Macular Assessment Profile (MAP) test, a VDU-based test incorporating the HFP technique. Foveal architecture was measured by optical coherence tomography. Safety parameters like CBC, RFT, LFT, RUA and UPT (for females of childbearing age) were also measured over a period of 180 days. The data obtained from 93 subjects were analyzed using ANCOVA.

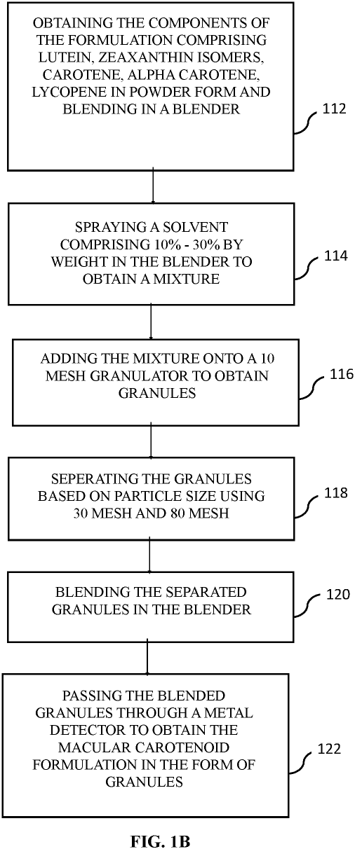

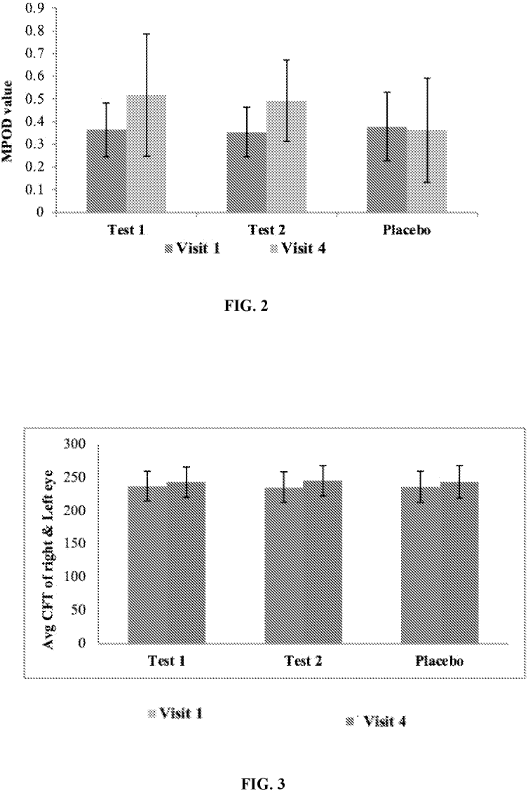

Results: Safety data demonstrated that all three study products did not alter any physiological functioning of the body and were considered safe for human use. Both the test products showed statistically significant improvements in the MPOD levels when compared from baseline to the last visit. On the other hand, the placebo arm did not show any significant change in MPOD levels. Similarly, when CFT was measured across all treatment groups, the test products showed statistically significant results in improving the mean CFT values from baseline compared to the placebo. Additionally, a direct correlation between MPOD and CFT for both the test products was observed, with r value being positive. FIG. 2 illustrates improvement in MPOD levels in subjects treated with test and placebo between Visit 1 to Visit 4, according to the aspect of the present invention. FIG. 3 illustrates graphical representation of improvement in Central Foveal Thickness (CFT), according to the aspect of the present invention.

Physical Examination: Every subject underwent detailed physical examination by the Investigator or his designee. All the subjects screened were completely normal with no abnormality and hence and only those subjects who were otherwise meeting the criteria of healthy as per the conditions in protocol were enrolled into the study.

Safety Parameters: Hematology and biochemistry parameters were found to be normal from visit 1 and last visit and there was NO clinically or statistically significant change observed in any of those parameters when compared with respective baseline values.

### Efficacy Parameters

### MPOD (Macular Pigment Optical Density)

In an embodiment, MPOD was studied across all three treatment groups. The mean MPOD values are statistically significant when compared with baseline (Visit 1), at p=0.0009 for Test product 1 (Lutein 5 mg + Zeaxanthin 1 mg) and p=0.0001 for Test product 2 (Lutein 5 mg + Zeaxanthin + Natural Mixed Carotenoids 5.5 mg), whereas it has not reached a statistically significant value when compared with baseline, at p=0.6179 for Placebo (Table 4.1). Between the two active arms, the MPOD values were more statistically significant in the arm containing the Natural Mixed Carotenoids than the arm containing just Lutein & Zeaxanthin, suggesting a synergistic effect of all the carotenoids. In table 4.1 Values are expressed as mean± SD. *p<0.05 when compared within the treatment groups from Visit 1 to Visit 4.

TABLE 4.1

Improvement in MPOD levels in subjects treated with test and placebo between Visit 1 to Visit 4

| Treatment | MPOD | | | | p-value^{$} (Within treatment) | p-value^{#} (Between treatment) |
|---|---|---|---|---|---|---|
| | N | Visit 1 | N | Visit 4 | | |
| Test 1 | 35 | 0.36±0.11 | 35 | 0.52±0.27^{$}* | 0.0009* | 0.0034* (Test 1 vs. Placebo) |
| Test 2 | 35 | 0.35±0.12 | 35 | 0.49±0.19^{$}* | 0.0001^{∗} | 0.0075* (Test 2 vs. Placebo) |
| Placebo | 23 | 0.38±0.15 | 23 | 0.36±0.23 | 0.6179 | N/AP |

### Central Foveal Thickness (CFT)

In the eye, a tiny pit located in the macula of the retina that provides the clearest vision of all is called fovea. Only in the fovea are the layers of the retina spread aside to let light fall directly on the cones, the cells that give the sharpest image. This is also called the central fovea or fovea centralis. In the present study, changes in the average CFT values (µm) of both eyes from visit 1 to visit 4 across the three treatment groups was observed. Increase of CFT values were seen in the two treatment groups that include subjects dosed with Test 1 (p=0.0156) and Test 2 (p=0.0031) as shown in Table 4.2. In table 4.2, values are expressed as mean± SD. *p<0.05 when compared within the treatment groups.

In the present study, CFT was measured across the three treatment groups and the observed mean CFT values were compared with baseline CFT values. These measured CFT values are depicted in the FIG. -2 where for Test product 1 (Lutein 10 mg + Zeaxanthin 2 mg), p=0.0156 and for Test product 2 (Lutein 10 mg + Zeaxanthin 2 mg + Natural Mixed Carotenoids), p=0.0031. Both Test products 1 and 2 produced statistically significant improvement in measured CFT values compared to the baseline. However, there was no statistical significance for placebo when compared with the baseline at p=0.1054.

TABLE 4.2

Inferential statistics for Average CFT of Left and Right Eyes

| Treatment | Average CFT of Left and Right Eyes | | | | p-value^{$} (Within treatment) | p-value^{#} (Between treatment) |
|---|---|---|---|---|---|---|
| | N | Visit 1 | N | Visit 4 | | |
| Test 1 | 35 | 237.6±22.82 | 35 | 243.3±22.94* | 0.0156* | 0.9306 (Test 1 vs. Placebo) |
| Test 2 | 35 | 235.9±22.76 | 35 | 245.4±23.23^{∗} | 0.0031* | 0.4755 (Test 2 vs. Placebo) |
| Placebo | 23 | 236.7±23.33 | 23 | 243.0±24.61 | 0.1054 | N/AP |

In the present study, CFT was measured across the three treatment groups and the observed mean CFT values were compared with baseline CFT values. These measured CFT values are depicted in the FIG. - 2 where for Test product 1 (Lutein 5 mg + Zeaxanthin 1 mg), p=0.0156 and for Test product 2 (Lutein 5 mg + Zeaxanthin + Natural Mixed Carotenoids 5.5 mg), p=0.0031. Both Test products 1 and 2 produced statistically significant improvement in measured CFT values compared to the baseline. However, there was no statistical significance for placebo when compared with the baseline at p=0.1054. Between the two active arms, the CFT values were more statistically significant in the arm containing the Natural Mixed Carotenoids than the arm containing just Lutein & Zeaxanthin, suggesting a synergistic effect of all the carotenoids.

The results obtained during the initial analysis of the formulation indicated positive correlation between MPOD and central foveal thickness levels, in healthy adult subjects.

### Correlation Between Central Foveal Thickness (CFT) and MPOD Levels in Healthy Adult Subjects

The results show that both test products aid in increasing the MPOD levels. Higher levels of MPOD may reduce the risk of Age-related Macular Degeneration (AMD), provide protection against oxidative damage, and protect against blue-light. Similarly, CFT values are seen increasing in both the test products. Improved CFT levels may increase and improve vision in normal individuals, as well as in patients with other concomitant conditions. This study strongly suggests that there is good correlation between MPOD and CFT levels compared to placebo.

TABLE 4.3

Correlation analysis on change in MPOD levels from Visit 1 to Visit 4 against change in CFT from Visit 1 to Visit 4

| Treatment | Variable | Simple Statistics | | Pearson Correlation Coefficient |
|---|---|---|---|---|
| | | N | Mean ± SD | |
| Test 1 | CFT (Change from Baseline to Visit 4) | 35 | 5.73±13.30 | r = 0.11459 p-value = 0.5122 |
| | MPOD (Change from Baseline to Visit 4) | 35 | 0.15±0.25 | |
| Test 2 | CFT (Change from Baseline to Visit 4) | 35 | 9.41±17.49 | r = 0.12896 p-value = 0.4603 |
| | MPOD (Change from Baseline to Visit 4) | 35 | 0.14±0.18 | |
| Placebo | CFT (Change from Baseline to Visit 4) | 23 | 6.27±17.81 | r = -0.00882 p-value = 0.9681 |
| | MPOD (Change from Baseline to Visit 4) | 23 | -0.02±0.15 | |

The observed data from the present study exhibited a significant positive correlation of MPOD and CFT in both the treatment groups. MPOD and CFT reached statistical significance amongst the two active treatment groups and Pearson correlation coefficient analysis showed r value of 0.12 (p=0.46) for Treatment 2. Similarly, r values for Treatment 1 and Placebo were 0.11 (p=0.51) and -0.0088 (p=0.98) respectively. These relationships are directly proportional and in line with the previously reported studies. The study showed that Test products were more significant in comparison to Placebo in improving the MPOD and CFT levels. Increased MPOD levels may benefit by reducing the risk of AMD, providing protection against oxidative damage and protecting eyes against bule-light. Similarly, improved CFT levels may increase and improve vision in normal individuals, and in subjects with other eye related concomitant conditions.

The formulation may be reconstituted into finished products and used as a nutrient, nutraceutical or dietary supplement and functional food. According to a further non-limiting exemplary aspect of the present invention the formulation can be incorporated into food products, such as cookies, cereals, crackers, doughnuts, bagels, biscuits, pasta, bread, baked goods, pizza dough, juices, gravies, sauces, salads, and candies.

The formulation of the present invention helps in increasing macular pigment optical density (MPOD) that helps to prevent age-related macular degeneration (ARMD).

The invention can be reconstituted into finished products for use as dietary supplements.

In further embodiments, the dietary supplement is a softgel capsules, two-piece hard-shell capsules, liquid-fill capsules, tablets, effervescent granules, gummies, powder mixes, stick packs, beverages, emulsions, bakery products, dairy products, tinctures or any combination thereof.

The present invention can be used for the development of functional food, dietary plan and as a nutritional supplement. In exemplary embodiment, formulation is available in forms like; oil suspension beadlets, powders, cold water-soluble powders, emulsions and granules.

According to a non-limiting exemplary aspect of the present invention can be used for the development of diet plan and nutritional supplements.

Merely for illustration, only representative number/type of graph, chart, block, and sub-block diagrams were shown. Many environments often contain many more block and sub-block diagrams or systems and sub-systems, both in number and type, depending on the purpose for which the environment is designed.

While specific embodiments of the invention have been shown and described in detail to illustrate the inventive principles, it will be understood that the invention may be embodied otherwise without departing from such principles.

Reference throughout this specification to “one embodiment”, “an embodiment”, or similar language means that a particular feature, structure, or characteristic described in connection with the embodiment is included in at least one embodiment of the present invention. Thus, appearances of the phrases “in one embodiment”, “in an embodiment” and similar language throughout this specification may, but do not necessarily, all refer to the same embodiment.

It should be understood that the figures and/or screen shots illustrated in the attachments highlighting the functionality and advantages of the present invention are presented for example purposes only. The present invention is sufficiently flexible and configurable, such that it may be utilized in ways other than that shown in the accompanying figures.

It should be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A macular carotenoid formulation for increasing the macular pigment optical density (MPOD) level in a subject, wherein the composition comprises:
a) Lutein in an amount of 2 to 20 mg, b) Zeaxanthin isomers in an amount of 0.4 to 4 mg, c) Beta Carotene in an amount of 1 to 10 mg, d) Alpha Carotene in an amount of 40 to 1000 mcg, e) Lycopene in an amount of 1 to 10 mg, and f) Beta Cryptoxanthin in an amount of 1 to 1000 mcg.

2. The macular carotenoid formulation of claim 1, wherein the macular carotenoid formulation is formulated using a carrier oil selected from sunflower oil, safflower oil, MCT (Medium Chain Triglycerides), castor oil, soybean oil, corn oil, olive oil, Omega-3 fatty acid oil or combinations thereof.

3. The macular carotenoid formulation of claim 1, wherein the formulation comprises at least one stabilizing agent selected from gum arabic, modified food starch, maltodextrin, cyclodextrin, sucrose, lecithin or combinations thereof.

4. The macular carotenoid formulation of claim 1, wherein the formulation is used as a nutrient, nutraceutical or dietary supplement.

5. A macular carotenoid formulation for increasing the macular pigment optical density (MPOD) level in a subject, wherein the composition comprises:
a) from about 1.66% to 16.6% by weight of Lutein, b) from about 0.34% to 3.4% by weight of Zeaxanthin-isomers, c) from about 0.74% to 8% by weight of Beta Carotene, d) from about 0.01% to 0.4% by weight of Alpha Carotene, e) from about 0.6% to 6.2% by weight of Lycopene, f) from about 0.001% to 0.1% by weight of Beta Cryptoxanthin, g) from about 0.01% to 2% by weight of Antioxidant, and h) from about 50% to 90% by weight of Carrier Oil.

6. The macular carotenoid formulation of claim 5, wherein the anti-oxidant is selected from vitamin E, mixed Vitamin E, mixed tocopherols or rosemary extract or combinations thereof, wherein the carrier oil is selected from sunflower oil, safflower oil, MCT (Medium Chain Triglycerides), castor oil, soybean oil, corn oil, olive oil, Omega-3 fatty acid oil or combinations thereof.

7. A macular carotenoid formulation for increasing the macular pigment optical density (MPOD) level in a subject, wherein the composition comprises:
a) from about 1.66% to 16.7% by weight of Lutein, b) from about 0.34% to 3.4% by weight of Zeaxanthin-isomers, c) from about 0.74% to 8% by weight of Beta Carotene, d) from about 0.01% to 0.4% by weight of Alpha Carotene, e) from about 0.6% to 6.2% by weight of Lycopene, f) from about 0.001% to 0.1% by weight of Beta Cryptoxanthin, g) from about 0.01% to 2% by weight of Antioxidant, h) from about 10% to 30% by weight of gum Arabic, and i) from about 25% to 75% by weight of a stabilizing agent.

8. The macular carotenoid formulation of claim 7, wherein the anti-oxidant is selected from vitamin E, mixed Vitamin E, mixed tocopherols or rosemary extract Or combinations thereof.

9. The macular carotenoid formulation of claim 7, wherein the stabilizing agent comprises at least one of a gum arabic, modified food starch, maltodextrin, cyclodextrin, sucrose, lecithin and combinations thereof.

10. The macular carotenoid formulation of claim 7, wherein the composition comprises a) 8.9% by weight of Lutein, b) 1.8% by weight of Zeaxanthin-isomers, c) 5.2% by weight of Beta Carotene, d) 0.3% by weight of Alpha Carotene, e) 3.1% by weight of Lycopene, f) 0.1%, by weight of Beta Cryptoxanthin, g) 1.0% by weight of Antioxidant, h) 20.5% by weight of gum Arabic, and i) 59.0% by weight of modified food starch.

11. The macular carotenoid formulation of claim 10, wherein the composition comprising the Lutein, the Zeaxanthin-isomers, the Beta Carotene, the Alpha Carotene, the Lycopene, the Beta Cryptoxanthin are stabilized in the gum arabic and/or the modified food starch along with the antioxidant selected from the group comprising vitamin E, mixed Vitamin E, mixed tocopherols and rosemary extract.

12. The macular carotenoid formulation of claim 1, wherein the formulation is formulated as soft gel capsules, two-piece hard-shell capsules, liquid-fill capsules, tablets, effervescent granules, gummies, powder mixes, stick packs, beverages, emulsions, bakery products, dairy products, tinctures, oil suspensions, bead-lets, powders, cold water-soluble powders, emulsions and granules or any combination thereof.

13. The composition of claim 1, wherein the subject is a mammal.

14. The composition of claim 1, wherein the subject is a human.

15. The method of preparing the macular carotenoid formulation of claim 1, wherein the macular carotenoid composition is derived from the vegetarian sources comprising xanthophylls, wherein the Lutein, Zeaxanthin and Meso Zeaxanthin are extracted from marigold flowers (Tagetes erecta), Alpha Carotene from carrot (Daucus carota subsp. sativus), the Beta Carotene is extracted from Blakeslea trispora, the Lycopene is extracted from Blakeslea trispora.

16. The method of claim 15, wherein the Beta Carotene is obtained from synthetic extraction.

17. The method of claim 15, wherein the Beta Carotene is extracted from Dunaliella salina, wherein the Alpha carotene is extracted from Elaeis guineensis.

18. The method of claim 15, wherein the Lycopene is extracted from Solanum lycopersicum.

19. The method of claim 15, wherein the Lutein, Zeaxanthin and Meso Zeaxanthin, Alpha carotene, Beta carotene and Lycopene are derived from the vegetarian sources using solvent extraction.

20. The method of claim 15, wherein the Lutein, Zeaxanthin and Meso Zeaxanthin, Alpha Carotene, Beta Carotene and Lycopene are derived from the vegetarian sources using super-critical extraction.
